(19) United States
(12) Plant Patent Application Publication
(10) Pub. No.: US 2014/0304873 P1
(43) Pub. Date: Oct. 9, 2014

(54) Coleus plant named Gator Glory

(50) ;

(71) Applicant: Florida Foundation Seed Producers, Inc., (US)

(72) Inventors: David G. Clark, Gainesville, FL (US); Grayson M. Clark, Gainesville, FL (US)

(21) Appl. No.: 13/986,164

(22) Filed: Apr. 5, 2013

(51) Int. Cl. A01H 5/00

(52) U.S. Cl. PLT/469

(57) ABSTRACT

‘Gator Glory’ is a new coleus plant distinguished by having bold orange leaves trimmed in bright yellow, novel growth habit, and desirable late-flowering characteristics, as disclosed.

### LATIN NAME OF THE GENUS AND SPECIES OF THE PLANT CLAIMED

Plectranthus scutellariodes

### CULTIVAR DENOMINATION

‘Gator Glory’

### BACKGROUND OF THE INVENTION

The invention relates to a new and distinct variety of coleus plant named ‘Gator Glory’. ‘Gator Glory’ originated from an open pollination conducted in May-November 2011 in Gainesville, Fla. between the female coleus plant ‘UF10-40-9’ (unpatented) and an unknown male coleus plant. A single seedling was chosen in May 2011 for further asexual propagation in Gainesville, Fla. (see FIG. 1 for pedigree).

‘Gator Glory’ has been reproduced asexually for over two years through vegetative cuttings and has been found to retain its distinctive characteristics through successive asexual propagations.

‘Gator Glory’ has not been made publicly available more than one year prior to the filing date of this application.

When ‘Gator Glory’ is compared to the female parent ‘UF10-40-9’ (unpatented), ‘Gator Glory’ has a bright orange foliage color with a bright yellow leaf margin, while ‘UF10-40-9’ has leaves colored deep maroon with bright yellow leaf margins.

When ‘Gator Glory’ is compared to the commercial cultivar ‘Rustic Orange’ (commercial, ProvenWinners Company), plants of ‘Rustic Orange’ have lighter orange foliage color and a much more upright growth habit (taller than wide), whereas ‘Gator Glory’ has a darker orange foliage color with more distinct yellow edges and a more spreading growth habit (wider than tall). ‘Rustic Orange’ also produces flowers earlier than ‘Gator Glory’, thus ‘Gator Glory’ retains more foliage over the course of a normal growing season in the garden.

### SUMMARY OF THE INVENTION

The following are the most outstanding and distinguishing characteristics of ‘Gator Glory’ when grown under normal horticultural practices in Gainesville, Fla. ‘Gator Glory’ has a combination of novel growth habit, late season flowering, excellent heat tolerance, and a consistent bright orange foliage color uniformly trimmed in yellow gold around the edges that is significantly different from other coleus plants. It has superior stability in foliage color in both sun and shade conditions, maintaining bright color in all conditions. It has an exceptional vigorous, mounded spreading growth habit with excellent lateral branching when grown as a stock plant, thus providing ample vegetative propagules for producers. This plant has not been observed to set a significant number of flowers in any trial to date, thus it is desirable for long-season performance in the landscape, as coleus plants that set seed usually experience late-season leaf drop.

### BRIEF DESCRIPTION OF THE DRAWINGS

This new coleus plant is illustrated by the accompanying photographs, which show the plant's form and foliage. The colors shown are as true as can be reasonably obtained by conventional photographic procedures. The photographs were taken from 3-month-old plants grown from cuttings in 1-gallon pots during December 2012-March 2013 in greenhouses in Gainesville, Fla.

FIG. 1—shows the pedigree of the claimed plant.

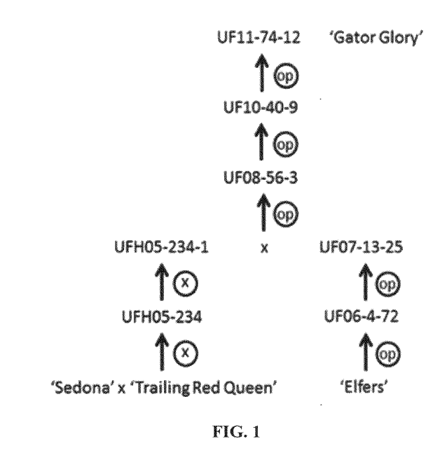

FIG. 2—shows the growth habit, form, and foliage of the claimed plant.

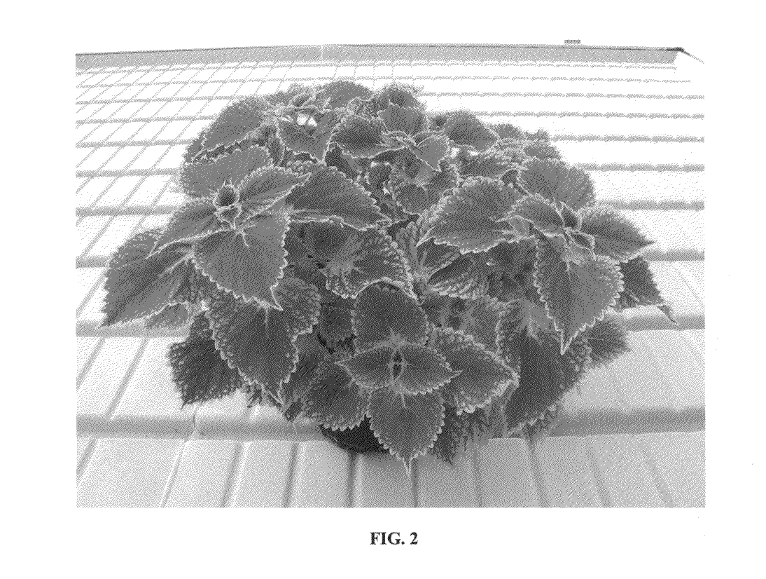

FIG. 3—shows a close-up of the foliage.

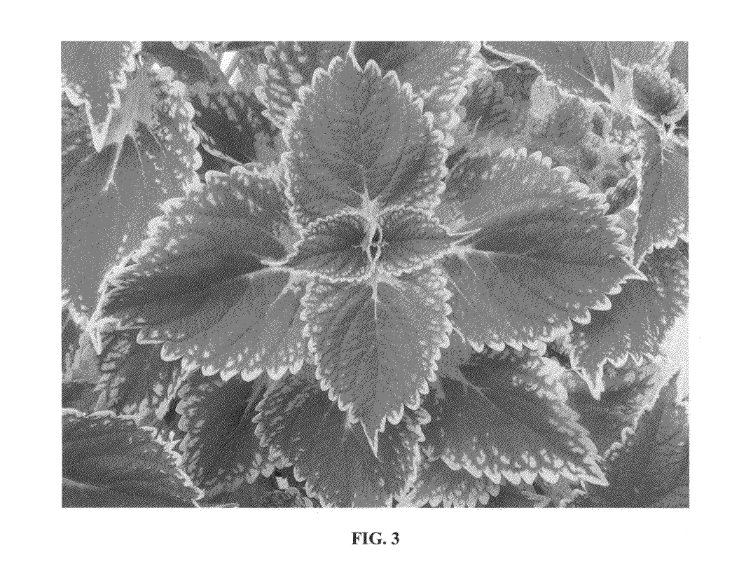

### DETAILED BOTANICAL DESCRIPTION

The following detailed description sets forth the distinctive characteristics of ‘Gator Glory’. The detailed description was obtained using 3-month-old plants from cuttings growing in a glass greenhouse in Gainesville, Florida in early spring 2013. The plants were pinched 2 weeks after cuttings were rooted, then grown in 1-gallon pots for approximately 10 weeks. Color references are to the RHS Colour Chart of The Royal Horticultural Society of London (R.H.S.), 2007 5th Edition.
Classification:
   Family.—Lamiaceae.
   Botanical.—Plectranthus scutellariodes.
   Common name.—Coleus.
   Cultivar name.—‘Gator Glory’.
Plant description:
   Form.—Spreading.
   Habit.—Mounded.
   Height (from top of soil).—26 cm.
   Width (horizontal plant diameter).—47 cm.
Propagation:
   Type cuttings.—Vegetative meristems having at least 1 node.
   Time to initiate roots.—3-4 days.
   Time to produce a rooted cutting.—7-10 days.
   Root habit.—Fibrous.
   Root description.—Callus forms in 2 to 3 days, roots initiate in 3-4 days and become a highly branched cutting in 7-10 days.
Branches:
   Quantity per plant.—5 main branches per plant with numerous side branches, pinched once.
   Branch color.—RHS 144C.
   Texture.—Smooth.
   Pubescence.—Non-descript.
   Stem description.—Square-shaped stem, 1.5 cm in diameter at the soil line.
   Branch diameter.—0.6 cm at the base of an 18-cm long branch.
   Branch length.—18 cm.
   Internode length.—1-4 cm.
   Anthocyanin.—N/A.
Leaves:
   Quantity of leaves per branch.—7-9. Arrangement: Opposite.
   Fragrance.—Not fragrant.
   Shape.—Ovate, consistent.
   Length.—9-10 cm.
   Width.—7-8 cm.
   Apex.—Broadly acuminate.
   Base.—Attenuate.
   Margin.—Sinuate.
   Leaf texture (both surfaces).—Slightly pubescent upper surface, smooth lower surface.
   Pubescence color (both surfaces).—Non-descript with naked eye.
   Venation color.—Upper surface: Base: RHS 144C; tip RHS N79B Lower surface: RHS 145B.
   Venation pattern.—Upper surface: Arcuate Lower surface: Reticulate.
   Color.—Immature leaf: Upper surface: RHS 172A in the center of the leaf, with RHS N144B margins Lower surface: RHS N79C in the center of the leaf, irregularly transitioning to RHS 199C toward the leaf edge.
   Color.—Mature leaf: Upper surface: RHS N34 in the center of the leaf, with RHS N144B margins Lower surface: RHS N79A in the center of the leaf, irregularly transitioning to RHS 147C toward the leaf edge.
   Petiole length.—2-6 cm.
   Petiole diameter.—0.2-0.3 cm.
   Petiole color.—RHS N144D.
Flowers and seeds: Flowers and seeds have not been observed.
Fruit/seed set: No fruit/seed observed
Disease and insect resistance: Disease and insect resistance is typical of the species, thus no claims are made of any superior disease or insect resistance with this cultivar. The most common insect pests observed on this plant in Gainesville, Fla. have been long-tailed or citrus mealybugs (Pseudococcus sp.), which occur on older stock plant material held in the greenhouse for over 3-4 months. Impatiens Necrotic Spot Virus (Bunyaviridae) has also been observed in plants confined in greenhouses with mixed crops (peppers) infected with Western flower thrips (Frankliniella occidentalis). The most common pathogen of this species in the U.S. is downy mildew (Pernonspora lamii). This pathogen has been observed in stock materials grown closely together in cooler growing seasons.

What is claimed is:

1. A new and distinct variety of Plectranthus scutellariodes called ‘Gator Glory’ as shown and described herein.
